(19) United States
(12) Patent Application Publication
(10) Pub. No.: US 2011/0082350 A1
(43) Pub. Date: Apr. 7, 2011

(54) ASSESSING A DEGREE OF VASCULAR BLOCKAGE OR RISK OF ISCHEMIA

(75) Inventor: Steve Koh, South Pasadena, CA (US)

(73) Assignee: PACESETTER, INC., Sylmar, CA (US)

(21) Appl. No.: 12/573,735

(22) Filed: Oct. 5, 2009

(51) Int. Cl. A61B 5/0205

(52) U.S. Cl. 600/301; 600/484

(57) ABSTRACT

A system and method for determining a patient's degree of cardiac vascular blockage or, equivalently, a patient's risk of cardiac ischemia, based on the time interval between the onset of exercise activity and the onset of an episode of cardiac ischemia. In one embodiment, an implantable cardiac device may obtain an EGM and possibly other measures of patient physiologic activity. These measures are used to determine when the patient has initiated exercise activity. Analysis of the EGM then detects an elevated or depressed ST segment, which typically indicates an episode of cardiac ischemia. The time interval between the onset of exercise and the onset of ischemia is a metric reflecting the patient's degree of vascular blockage or, equivalently, the patient's risk of ischemia. Other metrics may be derived, such as a substantially workload-level invariant measure determined as the product of the exercise workload level and the ischemia onset time interval.

### CROSS REFERENCE TO RELATED APPLICATIONS

This application is related to commonly owned, co-pending U.S. application Ser. No. 11/611,105 to Steve Koh, filed Dec. 14, 2006, entitled “Exercise Compliance Monitoring and Benefit Assessment via a Composite Physiologic Signal Determined by Implanted Physiologic Sensor,” the disclosure of which is incorporated herein by reference as though set forth in full below.

### FIELD OF THE INVENTION

The present invention relates generally to implantable cardiac devices and, more particularly, to methods and systems for determining a degree of cardiac coronary blockage or risk of ischemia using an implantable cardiac device.

### BACKGROUND

Myocardial ischemia is a cardiac function disorder wherein there exists insufficient blood flow to the muscle tissue of the heart, most commonly due to narrowing of the coronary arteries. Ischemia may lead to necrosis of cardiac muscle, especially if the narrowing of the arteries is severe or if an ischemic episode is sufficiently prolonged. Conventionally, an episode of myocardial ischemia may be diagnosed by monitoring changes in an electrocardiogram (e.g., a surface electrocardiogram (ECG), or an internal electrogram (EGM) obtained by leads implanted in the heart). The pattern of cardiac electrical activity shown on an ECG or EGM is conventionally labeled with letters of the alphabet corresponding to various peaks and valleys, e.g., ‘P’, ‘Q’, ‘R’, ‘S’, and ‘T’. The segments or intervals connecting these peaks and valleys are conventionally labeled as the ‘PR interval’, ‘PR segment’, ‘QRS segment’, etc.

Myocardial ischemia is typically diagnosed based on abnormalities detected in the ST segment of an ECG or EGM. In particular, an elevated ST segment in an ECG is typically indicative of an episode of myocardial ischemia. However, particularly in an EGM, in some instances it may be that ST segment depression (i.e., a lowering of the level of the ST segment) may be indicative of ischemia. This is discussed further below.

In a resting patient or patient engaged in moderate activity, even a patient with significant narrowing of the coronary arteries, there may be no detectable signs of ischemia. In other words, there may be no symptoms of oxygen insufficiency, and an ECG or EGM may not indicate any abnormalities in the ST segment. As a result, narrowing of the arteries may not become apparent until severe coronary artery blockage has developed, at which point the patient's health may be significantly compromised. In some cases, cardiac ischemia may not be symptomatic or may be difficult to even detect until the patient is experiencing a coronary episode, for example, during unusually heavy exercise such as shoveling snow. Such undetected ischemia may even precipitate a heart attack. The detection of ST segment elevation during emergency medical treatment may be too late for prophylactic measures (e.g., dietary changes, exercise, cholesterol-reducing medicines, etc.) to prevent or control coronary disease.

What is needed, then, is a method and system of ongoing monitoring to detect a degree of vascular blockage prior to the onset of severe cardiac impairment. In the event a patient has already experienced a major coronary episode and has received treatment (for example, by-pass surgery), what is needed is a means of ongoing monitoring to ensure that vascular blockage does not return; or that if it does return, it can be detected early enough for treatment to prevent another major coronary episode.

### BRIEF SUMMARY

Methods and systems are presented to determine a degree of coronary vascular blockage or, equivalently, a degree of risk of cardiac ischemia. In an exemplary embodiment, an implantable cardiac device (ICD) is used to monitor patient cardiac activity while the patient is engaged in exercise. Data obtained from the ICD may be used both to determine when the patient has begun exercising, and also to determine the onset of ST segment elevation or ST segment depression, which may be indicative of cardiac ischemia. The time interval between the onset of exercise and the onset of ST segment elevation or ST segment depression may be used as a metric to indicate a degree of coronary blockage, wherein a shorter time interval may reflect a higher degree of coronary vascular blockage. A combined metric based on both the level of exercise activity and the onset interval for ST segment elevation or depression may also be used.

It should be noted that throughout this document, reference is made both to “a method or system of assessing or determining a degree of cardiac vascular blockage”, and also to “a method or system of assessing a degree of risk of cardiac ischemia”. Reference is also made to “a measure of ischemia susceptibility”. For purposes of the present method and system, a “degree of cardiac vascular blockage”, a “degree of risk of cardiac ischemia”, a “measure of ischemia susceptibility” and similar terms are considered to be synonymous concepts, and the indicated phrases and substantially analogous phrases are used interchangeably throughout.

Further features and advantages of the methods and systems presented herein, as well as the structure and operation of various example methods and systems, are described in detail below with reference to the accompanying drawings.

### BRIEF DESCRIPTION OF THE DRAWINGS/FIGURES

The accompanying drawings, which are incorporated herein and form part of the specification, illustrate the methods and systems presented herein for assessing a degree of vascular blockage by measuring the time between onset of workload and onset of ST segment elevation or ST segment depression. Together with the description, the drawings further serve to explain the principles of and to enable a person skilled in the relevant art(s) to make and use the methods and systems presented herein. In the drawings, like reference numbers indicate identical or functionally similar elements. Further, the drawing in which an element first appears is typically indicated by the leftmost digit(s) in the corresponding reference number (e.g., an element numbered 302 first appears in FIG. 3).

FIG. 1 is a simplified diagram illustrating an example implantable cardiac device (ICD) in electrical communication with a patient's heart by means of three leads suitable for delivering multi-chamber stimulation and pacing therapy and for detecting cardiac electrical activity.

FIG. 2 is a functional block diagram of an example ICD that can detect cardiac electrical activity and analyze cardiac electrical activity, as well as provide cardioversion, defibrillation, and pacing stimulation in four chambers of a heart.

FIGS. 3A and 3B illustrate an electrogram (EGM) or electrocardiogram (ECG) indicative of healthy cardiac function and an EGM or ECG indicative of an episode of cardiac ischemia, respectively.

FIG. 4 is a process flowchart for an exemplary method for assessing a degree of cardiac vascular blockage or, equivalently, a degree of risk of cardiac ischemia, by measuring the time between onset of workload or patient exercise activity and onset of ST segment elevation which is indicative of myocardial ischemia.

FIGS. 5A, 5B, and 5C each provide a graphical illustration of exemplary methods of determining the time between the onset of patient workload or exercise activity and the time of onset of ST segment elevation.

FIGS. 6A and 6B illustrate an exemplary method for calculating a measure of ischemia susceptibility which is substantially invariant with respect to the exercise workload imposed on the patient at the time the measure is obtained.

FIGS. 7A and 7B illustrate exemplary analysis processes by which assessments may be made of a degree of vascular blockage for a patient, based on prior historical data.

FIG. 7C illustrates a comparison between an exemplary ideal curve of substantially work-load invariant ischemia susceptibility and an exemplary curve based on data points which cluster about the ideal curve.

FIG. 8A and FIG. 8B illustrate an exemplary method of assessing a long term change in vascular blockage or ischemia risk for a patient.

### DETAILED DESCRIPTION

### 1. Overview

The following detailed description of methods and systems for assessing a degree of vascular blockage by measuring the time between onset of workload and onset of ST segment elevation or ST segment depression refers to the accompanying drawings that illustrate exemplary embodiments consistent with these methods and systems. Other embodiments are possible, and modifications may be made to the embodiments within the spirit and scope of the methods and systems presented herein. Therefore, the following detailed description is not meant to limit the methods and systems described herein. Rather, the scope of these methods and systems is defined by the appended claims.

It would be apparent to one of skill in the art that the methods and systems for assessing a degree of vascular blockage by measuring the time between onset of workload and onset of ST segment elevation or ST segment depression, as described below, may be implemented in many different embodiments of hardware, software, firmware, and/or the entities illustrated in the figures. Any actual software and/or hardware described herein is not limiting of these methods and systems. Thus, the operation and behavior of the methods and systems will be described with the understanding that modifications and variations of the embodiments are possible, given the level of detail presented herein.

In particular, while the methods and systems herein are described using an exemplary embodiment which may employ an implantable cardiac device (ICD) to measure cardiac electrical activity and perform an EGM, the methods and systems described herein for assessing a degree of vascular blockage may also be implemented via an external electrocardiograph which provides an ECG, or may also be implemented via other hardware and software configurations which may be partially external and partially internal to the patient.

Before describing in detail the methods and systems for assessing a degree of vascular blockage by measuring the time between onset of workload and onset of ST segment elevation or ST segment depression, it is helpful to describe an example environment in which these methods and systems may be implemented. The methods and systems described herein may be particularly useful in the environment of an ICD.

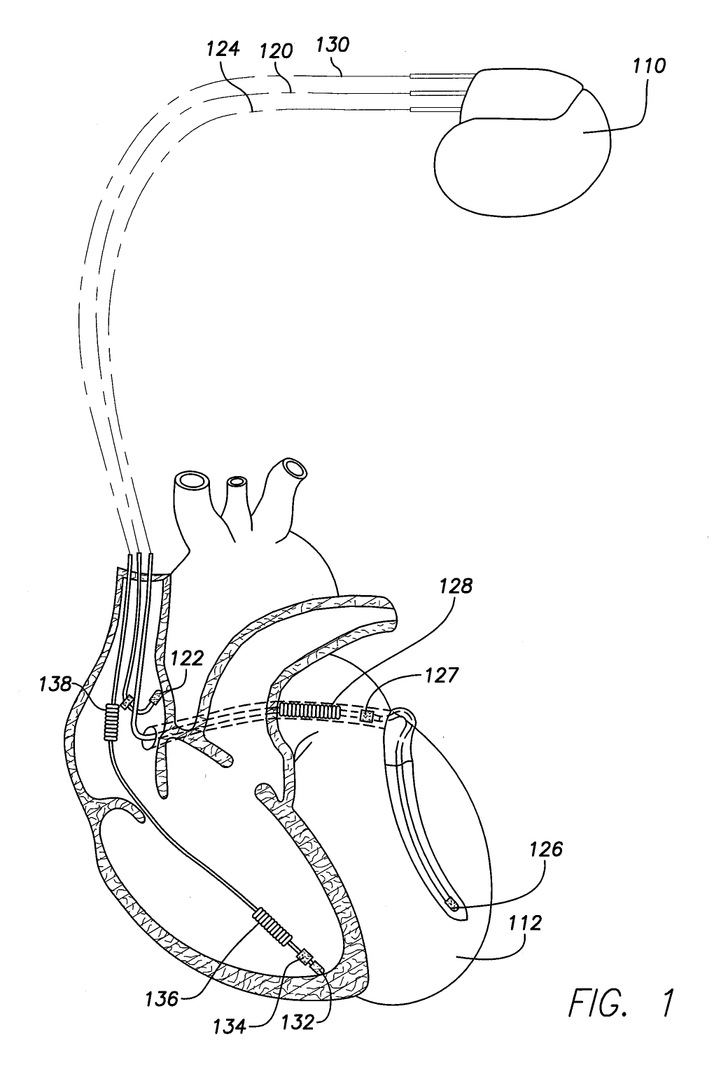

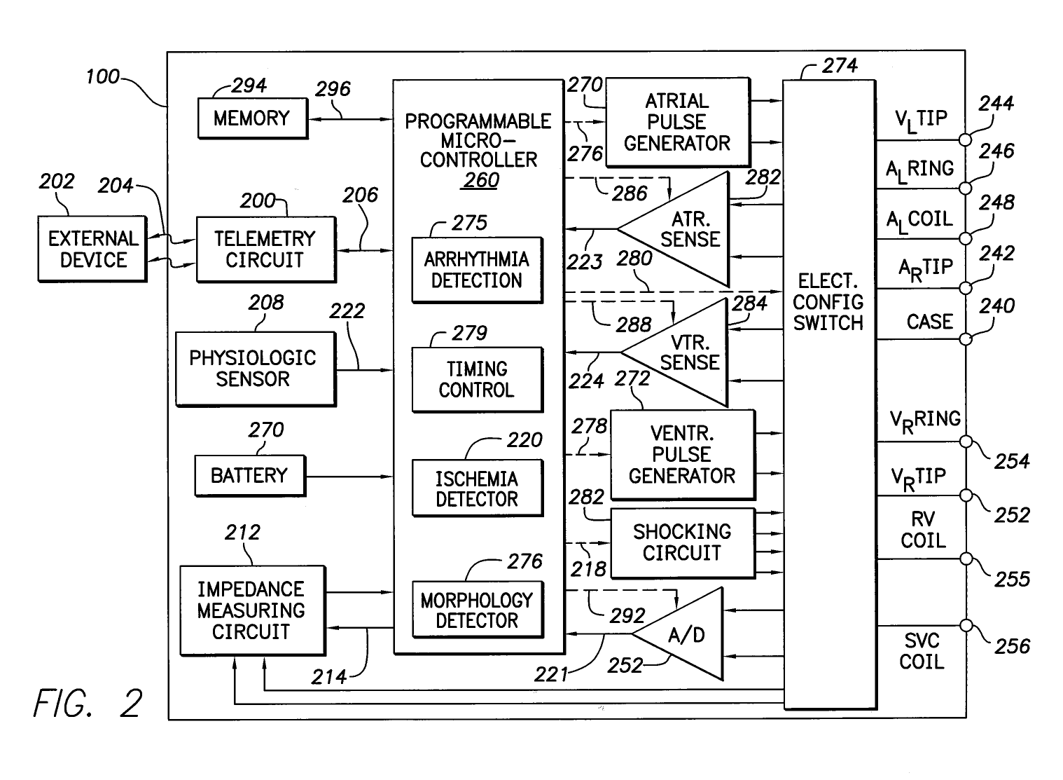

An ICD is a physiologic measuring device that is implanted in a patient to monitor cardiac function and to deliver appropriate electrical therapy, for example, pacing pulses, cardioverting and defibrillator pulses, and drug therapy, as required. ICDs include, for example, pacemakers, cardioverters, defibrillators, implantable cardioverter defibrillators, implantable cardiac rhythm management devices, and the like. Such devices may also be used in particular to monitor cardiac electrical activity and to analyze cardiac electrical activity. The term “implantable cardiac device” or simply “ICD” is used herein to refer to any implantable cardiac device. FIGS. 1 and 2 illustrate such an environment which may be used to implement the methods and systems described herein for detecting a degree of cardiac vascular blockage or a risk of ischemia.

### 2. Exemplary ICD in Electrical Communication with a Patient's Heart

FIG. 1 illustrates an exemplary ICD 110 in electrical communication with a patient's heart 112 by way of three leads, 120, 124 and 130, suitable for delivering multi-chamber stimulation and pacing therapy. To sense atrial cardiac signals and to provide right atrial chamber stimulation therapy, ICD 110 is coupled to implantable right atrial lead 120 having at least an atrial tip electrode 122, which typically is implanted in the patient's right atrial appendage.

To sense left atrial and ventricular cardiac signals and to provide left-chamber pacing therapy, ICD 110 is coupled to “coronary sinus” lead 124 designed for placement in the “coronary sinus region” via the coronary sinus for positioning a distal electrode adjacent to the left ventricle and/or additional electrode(s) adjacent to the left atrium. As used herein, the phrase “coronary sinus region” refers to the vasculature of the left ventricle, including any portion of the coronary sinus, great cardiac vein, left marginal vein, left posterior ventricular vein, middle cardiac vein, and/or small cardiac vein or any other cardiac vein accessible by the coronary sinus.

Accordingly, exemplary coronary sinus lead 124 is designed to receive atrial and ventricular cardiac electrical signals and to deliver left ventricular pacing therapy using at least a left ventricular tip electrode 126, left atrial pacing therapy using at least a left atrial ring electrode 127, and shocking therapy using at least a left atrial coil electrode 128.

ICD 110 is also shown in electrical communication with the patient's heart 112 by way of implantable right ventricular lead 130 having, in this embodiment, a right ventricular tip electrode 132, a right ventricular ring electrode 134, a right ventricular (RV) coil electrode 136, and a superior vena cava (SVC) coil electrode 138. Typically, right ventricular lead 130 is transvenously inserted into heart 112 so as to place right ventricular tip electrode 132 in the right ventricular apex so that RV coil electrode 136 will be positioned in the right ventricle and SVC coil electrode 138 will be positioned in the SVC.

Accordingly, exemplary right ventricular lead 130 is capable of receiving cardiac electrical signals, as well as delivering stimulation in the form of pacing and shock therapy to the right ventricle.

### 3. Functional Elements of an Exemplary ICD

FIG. 2 shows a simplified block diagram of ICD 110, which is capable of treating both fast and slow arrhythmias with stimulation therapy, including cardioversion, defibrillation, and pacing stimulation; ICD 110 is also capable of detecting cardiac electrical signals, and in particular is capable of detecting myocardial ischemia via detection of an elevated or depressed (i.e., lowered) ST segment. While a particular multi-chamber device is shown, it is shown for illustration purposes only, and one of skill in the art could readily duplicate, eliminate or disable the appropriate circuitry in any desired combination to provide a device capable of detecting suitable cardiac electrical signals for a variety of cardiac monitoring, health assessment, and health maintenance purposes including, for example and without limitation, performing an EGM, detecting ST segment elevation or depression in an EGM, and detecting cardiac ischemia.

A housing 240 of ICD 110, shown schematically in FIG. 2, is often referred to as the “can,” “case” or “case electrode” and may be programmably selected to act as the return electrode for all “unipolar” modes. Housing 240 may further be used as a return electrode for shocking purposes alone or in combination with one or more of coil electrodes, 128, 136, and 138, which are shown in FIG. 1. Housing 240 further includes a connector (not shown) having a plurality of terminals, 242, 244, 246, 248, 252, 254, 256, and 258 (shown schematically and, for convenience, the names of the electrodes to which they are connected are shown next to the terminals). As such, to achieve right atrial sensing and pacing, the connector includes at least a right atrial tip terminal (AR TIP) 242 adapted for connection to atrial tip electrode 122 (shown in FIG. 1).

To achieve left chamber sensing, pacing and shocking, the connector includes at least a left ventricular tip terminal (VL TIP) 244, a left atrial ring terminal (AL RING) 246, and a left atrial shocking terminal (AL COIL) 248, which are adapted for connection to left ventricular ring electrode 126, left atrial tip electrode 127, and left atrial coil electrode 128 (all shown in FIG. 1), respectively.

To support right chamber sensing, pacing, and shocking the connector also includes a right ventricular tip terminal (VR TIP) 252, a right ventricular ring terminal (VR RING) 254, a right ventricular shocking terminal (RV COIL) 256, and an SVC shocking terminal (SVC COIL) 258, which are configured for connection to right ventricular tip electrode 132, right ventricular ring electrode 134, RV coil electrode 136, and SVC coil electrode 138 (all shown in FIG. 1), respectively.

At the core of ICD 110 is a programmable microcontroller 260, which may control the various modes of stimulation therapy and may also control the collection and analysis of cardiac electrical activity data. As is well known in the art, microcontroller 260 typically includes a microprocessor, or equivalent control circuitry, designed specifically for controlling the delivery of stimulation therapy and for the analysis of cardiac electrical activity, and can further include RAM or ROM memory, logic and timing circuitry, state machine circuitry, and I/O circuitry. Typically, microcontroller 260 includes the ability to process or monitor input signals (including, but not limited to, data concerning cardiac electrical activity) as controlled by a program code stored in a designated block of memory.

The details of the design of microcontroller 260 may not be critical to the techniques presented herein. Rather, any suitable microcontroller 260 can be used to carry out the functions described herein. The use of microprocessor-based control circuits for performing timing and data analysis functions are well known in the art. Microcontroller 260 may include dedicated hardware, firmware, or software for the analysis of cardiac electrical signals.

Representative types of control circuitry that may be used with the techniques presented herein include the microprocessor-based control system of U.S. Pat. No. 4,940,052 (Mann et. al.) and the state-machines of U.S. Pat. Nos. 4,712,555 (Thornander et al.) and 4,944,298 (Sholder). For a more detailed description of the various timing intervals used within ICDs and their inter-relationship, see U.S. Pat. No. 4,788,980 (Mann et. al.). The '052, '555, '298 and '980 patents are incorporated herein by reference.

As shown in FIG. 2, an atrial pulse generator 270 and a ventricular pulse generator 272 generate pacing stimulation pulses for delivery by right atrial lead 120, right ventricular lead 130, and/or coronary sinus lead 124 (shown in FIG. 1) via an electrode configuration switch 274. It is understood that in order to provide stimulation therapy in each of the four chambers of the heart, atrial and ventricular pulse generators 270 and 272 may include dedicated, independent pulse generators, multiplexed pulse generators, or shared pulse generators. Pulse generators 270 and 272 are controlled by microcontroller 260 via appropriate control signals 276 and 278, respectively, to trigger or inhibit the stimulation pulses.

Microcontroller 260 further includes timing control circuitry 279, which is used to control pacing parameters (e.g., the timing of stimulation pulses) as well as to keep track of the timing of refractory periods, post ventricular atrial refractory period (PVARP) intervals, noise detection windows, evoked response windows, alert intervals, marker channel timing, etc., which are well known in the art. Examples of pacing parameters include, but are not limited to, atrioventricular (AV) delay, interventricular (RV-LV) delay, atrial interconduction (A-A) delay, ventricular interconduction (V-V) delay, and pacing rate. Timing control circuitry 279 may also be used to determine the duration of cardiac events, or be used to determine the duration of intervals in a cardiac EGM, such as the duration of the ST segment. Timing control circuitry 279 may also provide other timing information which is useful in the analysis of an EGM.

Switch 274 includes a plurality of switches for connecting the desired electrodes to the appropriate I/O circuits, thereby providing complete electrode programmability. Accordingly, switch 274, in response to a control signal 280 from microcontroller 260, determines the polarity of the stimulation pulses (e.g., unipolar, bipolar, combipolar, etc.) by selectively closing the appropriate combination of switches (not shown) as is known in the art. Switch 274, in response to a control signal 280 from microcontroller 260, may also route cardiac electrical signals to appropriate analysis circuitry in microcontroller 260.

In particular, atrial sensing (ATR. SENSE) circuits 282 and ventricular sensing (VTR. SENSE) circuits 284 may also be selectively coupled to right atrial lead 120, coronary sinus lead 124, and right ventricular lead 130, which are shown in FIG. 1, through switch 274 for detecting the presence of cardiac activity in each of the four chambers of the heart. Accordingly, atrial and ventricular sensing circuits 282 and 284 may include dedicated sense amplifiers, multiplexed amplifiers, or shared amplifiers. Switch 274 determines the “sensing polarity” of the cardiac signal by selectively closing the appropriate switches, as is also known in the art. In this way, a clinician may program the sensing polarity independent of the stimulation polarity.

Each sensing circuit, 282 and 284, preferably employs one or more low power, precision amplifiers with programmable gain and/or automatic gain control, bandpass filtering, and a threshold detection circuit, as known in the art, to selectively sense the cardiac signal of interest. The automatic gain control enables ICD 110 to deal effectively with the difficult problem of sensing the low amplitude signal characteristics of atrial or ventricular fibrillation. Such sensing circuits, 282 and 284, can be used to determine cardiac performance values used in the techniques presented herein.

The outputs of atrial and ventricular sensing circuits 282 and 284 are connected to microcontroller 260 which, in turn, are able to trigger or inhibit atrial and ventricular pulse generators, 270 and 272, respectively, in a demand fashion in response to the absence or presence of cardiac activity, in the appropriate chambers of the heart. Sensing circuits 282 and 284, in turn, receive control signals over signal lines 286 and 288 from microcontroller 260 for purposes of measuring cardiac performance at appropriate times, and for controlling the gain, threshold, polarization charge removal circuitry (not shown), and timing of any blocking circuitry (not shown) coupled to the inputs of sensing circuits 282 and 286.

For arrhythmia detection, ICD 110 utilizes the atrial and ventricular sensing circuits 282 and 284 to sense cardiac signals to determine whether a rhythm is physiologic or pathologic. The timing intervals between sensed events (e.g., P-waves, R-waves, and depolarization signals associated with fibrillation, which are sometimes referred to as “F-waves” or “Fib-waves”) are then classified by microcontroller 260 by comparing them to a predefined rate zone limit (i.e., bradycardia, normal, low rate ventricular tachycardia (VT), high rate VT, and fibrillation rate zones) and various other characteristics (e.g., sudden onset, stability, physiologic sensors, and morphology, etc.) in order to determine the type of remedial therapy that is needed (e.g., bradycardia pacing, anti-tachycardia pacing, cardioversion shocks or defibrillation shocks, collectively referred to as “tiered therapy”).

Microcontroller 260 utilizes arrhythmia detection circuitry 275 and morphology detection circuitry 276 to recognize and classify arrhythmia so that appropriate therapy can be delivered.

In an embodiment, microcontroller 260 utilizes ischemia detection circuitry 220 for monitoring cardiac electrical activity to diagnose myocardial ischemia, such as by monitoring for ST segment elevation or ST segment depression in an EGM. Ischemia detection circuitry 220 may also monitor for cardiac heart rate data as well.

Cardiac signals are also applied to the inputs of an analog-to-digital (ND) data acquisition system 290. Data acquisition system 290 is configured to acquire EGM signals, convert the raw analog data into a digital signal, and store the digital signals for later processing and/or telemetric transmission to an external device 202. Data acquisition system 290 is coupled to right atrial lead 120, coronary sinus lead 124, and right ventricular lead 130, which are shown in FIG. 1, through switch 274 to sample cardiac signals across any pair of desired electrodes.

Advantageously, data acquisition system 290 can be coupled to microcontroller 260, or other detection circuitry, for detecting an evoked response from heart 112 in response to an applied stimulus, thereby aiding in the detection of “capture.” Capture occurs when an electrical stimulus applied to the heart is of sufficient energy to depolarize the cardiac tissue, thereby causing the heart muscle to contract. Microcontroller 260 detects a depolarization signal during a window following a stimulation pulse, the presence of which indicates that capture has occurred. Microcontroller 260 enables capture detection by triggering ventricular pulse generator 272 to generate a stimulation pulse, starting a capture detection window using timing control circuitry 279 within microcontroller 260, and enabling data acquisition system 290 via a control signal 292 to sample the cardiac signal that falls in the capture detection window and, based on the amplitude, determines if capture has occurred.

The implementation of capture detection circuitry and algorithms are well known. See for example, U.S. Pat. No. 4,729,376 (Decote, Jr.); U.S. Pat. No. 4,708,142 (Decote, Jr.); U.S. Pat. No. 4,686,988 (Sholder); U.S. Pat. No. 4,969,467 (Callaghan et. al.); and U.S. Pat. No. 5,350,410 (Kleks et al.), which patents are hereby incorporated herein by reference. The type of capture detection system used is not critical to the techniques presented herein.

Microcontroller 260 is further coupled to a memory 294 by a suitable data/address bus 296. The programmable operating parameters used by microcontroller 260 are stored and modified, as required, in memory 294 in order to customize the operation of ICD 110 to suit the needs of a particular patient. Such operating parameters may define, for example, the exact physiologic or biometric parameters which indicate that patient exercise has commenced, while other stored parameters may determine the nature or degree of ST segment elevation or ST segment depression which is taken as indicative of an episode of ischemia.

Advantageously, the operating parameters of ICD 110 may be non-invasively programmed into memory 294 through a telemetry circuit 200 in telemetric communication with external device 202, such as a programmer, transtelephonic transceiver, or a diagnostic system analyzer. Telemetry circuit 200 is activated by microcontroller 260 by a control signal 206. Telemetry circuit 200 advantageously allows EGMs and status information relating to the operation of ICD 110 (as contained in microcontroller 260 or memory 294) to be sent to external device 202 through an established communication link 204. Telemetry circuit 200 also allows data obtained by an external sensor device to be passed to microcontroller 260 for analysis.

For examples of such devices, see U.S. Pat. No. 4,809,697, entitled “Interactive Programming and Diagnostic System for use with Implantable Pacemaker” (Causey, III et al.); U.S. Pat. No. 4,944,299, entitled “High Speed Digital Telemetry System for Implantable Device” (Silvian); and U.S. Pat. No. 6,275,734, entitled “Efficient Generation of Sensing Signals in an Implantable Medical Device such as a Pacemaker or ICD” (McClure et al.), which patents are hereby incorporated herein by reference.

ICD 110 further includes a physiologic sensor 208 that can be used to detect changes in cardiac performance or changes in the physiological condition of the heart. Accordingly, microcontroller 260 can respond by adjusting the various pacing parameters (such as rate, AV Delay, RV-LV Delay, V-V Delay, etc.). Microcontroller 260 controls adjustments of pacing parameters by, for example, controlling the stimulation pulses generated by the atrial and ventricular pulse generators 270 and 272. While shown as being included within ICD 110, it is to be understood that physiologic sensor 208 may also be external to ICD 110, yet still be implanted within or carried by the patient. More specifically, sensor 208 can be located inside ICD 110, on the surface of ICD 110, in a header of ICD 110, or on a lead (which can be placed inside or outside the bloodstream).

In an embodiment, physiologic sensor 208 can include an implantable, intra-ventricular pressure transducer that generates a signal indicative of ventricular cardiac pressure, characteristics of which may be monitored by ischemia detector 220 to diagnose myocardial ischemia. Such a pressure transducer, with associated ischemia detection capability, may supplement or augment the ischemia detection capability obtained via a determination of ST segment elevation or depression (the latter means of detection being described in greater detail later in this document).

Physiologic sensor 208 is not limited to pressure transducers, and can include other types of sensors capable of generating a signal indicative of ventricular cardiac pressure, such as strain gauge sensors, photoplethysmography (PPG) sensors, and the like. In turn, these technologies may provide data from which may be determined the patient heart rate, patient respiration rate, patient instantaneous blood pressure, or related physiologic parameters. Physiologic sensor 208 may also comprise a means for detecting patient blood oxygen concentration level (SVO₂).

In some cases, determining some physiologic values may require calculations based on data obtained via the electronics technologies already described above within ICD 110. For example, a patient heart rate signal may be determined by monitoring peaks in an EGM obtained via ICD 110, or by means of other sensors within ICD 110. The patient respiration rate may then be obtained via a low-pass filtering of the patient heart rate signal. Or, both patient heart rate and respiration rate may be derived values obtained via analysis or filtering of direct measurements of patient instantaneous blood pressure.

Commonly owned, co-pending U.S. application Ser. No. 11/611,105 to Steve Koh, filed Dec. 14, 2006, entitled “Exercise Compliance Monitoring And Benefit Assessment Via A Composite Physiologic Signal Determined By Implanted Physiologic Sensor”, incorporated herein by reference in its entirety, teaches a number of suitable technologies for obtaining various physiologic signals or values via ICD 110, and for deriving other physiologic signals or values from the measured signals or values.

Further, ICD 110 may include an accelerometer (not shown) or other means to detect motion or acceleration, as means or as partial means to determine if a patient is exercising or otherwise engaged in physical activity.

ICD 110 further includes a magnet detection circuitry (not shown), coupled to microcontroller 260. It is the purpose of the magnet detection circuitry to detect when a magnet is placed over ICD 110. A clinician may use the magnet to perform various test functions of ICD 110 and/or to signal microcontroller 260 that external device 202 is in place to receive or transmit data to microcontroller 260 through telemetry circuit 200.

As further shown in FIG. 2, ICD 110 is shown as having an impedance measuring circuit 212, which is enabled by microcontroller 260 via a control signal 214. The known uses for an impedance measuring circuit 212 include, but are not limited to, lead impedance surveillance during the acute and chronic phases for proper lead positioning or dislodgement; detecting operable electrodes and automatically switching to an operable pair if dislodgement occurs; measuring respiration or minute ventilation; measuring thoracic impedance for determining shock thresholds; detecting when the device has been implanted; measuring stroke volume; and detecting the opening of heart valves, etc. Impedance measuring circuit 212 is advantageously coupled to switch 274 so that any desired electrode may be used. Impedance measuring circuit 212 is not critical to the techniques presented herein and is shown only for completeness.

In the case where ICD 110 is intended to operate as a cardioverter, pacer or defibrillator, it must detect the occurrence of an arrhythmia and automatically apply an appropriate electrical therapy to the heart aimed at terminating the detected arrhythmia. To this end, microcontroller 260 further controls a shocking circuit 216 by way of a control signal 218. Shocking circuit 216 generates shocking pulses of low (e.g., up to 0.5 Joules), moderate (e.g., 0.5-10 Joules), or high energy (e.g., 11-40 Joules), as controlled by microcontroller 260. Such shocking pulses are applied to the patient's heart 112 through at least two shocking electrodes (e.g., selected from left atrial coil electrode 128, RV coil electrode 136, and SVC coil electrode 138, which are shown in FIG. 1). As noted above, housing 240 may act as an active electrode in combination with RV electrode 136, or as part of a split electrical vector using SVC coil electrode 138 or left atrial coil electrode 128 (i.e., using the RV electrode as a common electrode).

As will be discussed further below, the present method for determining a degree of cardiac vascular blockage or, equivalently, determining a risk of cardiac ischemia, may rely on determining when a patient is engaged in exercise activity. Such a determination may be made by determining when various measures of patient metabolic or physiologic activity have crossed appropriate metabolic thresholds or physiologic thresholds, or determining when patient movement has crossed a movement threshold. For example, a determination may be made that patient heart rate, respiration rate, or blood pressure have exceeded respective heart rate, respiration rate, or blood pressure thresholds.

Similarly, a determination may be made that patient movement has exceeded a movement threshold. In some cases, alternate or supplementary criteria based on external activity measurements may be employed, such as the number of minutes spent on a treadmill or similar exercise device at a specified level of difficulty, wherein an external activity threshold or an exercise activity duration threshold is employed. Some combination of these threshold criteria (i.e., metabolic thresholds, movement thresholds, activity duration thresholds, and/or external activity thresholds) may also be employed to determine that patient exercise activity has been initiated, or initiated at a satisfactory level of physical workload.

Some or all of these metabolic measures or movement measures, or other measures which reflect a level of patient exercise or patient activity, may be obtained via ICD 110. ICD 110 may also have the necessary software, firmware, or hardware to determine when the respective metabolic, movement, or other exercise or activity thresholds have been exceeded, thus indicating patient exercise.

ICD 110 additionally includes a battery 210, which provides operating power to a load that includes all of the circuits shown in FIG. 2.

### 4. Determination of an Episode of Cardiac Ischemia via Elevated ST Segment or Depressed ST Segment

Myocardial ischemia is a condition where there is an inadequate supply of blood to the heart, resulting in an insufficient supply of oxygen for adequate oxygenation of the cardiac muscle. Prolonged ischemia, or ischemia of shorter duration but of sufficient severity, may result in damage to the cardiac tissue. Ischemia is typically caused by partial or complete blockage of one or more of the coronary arteries.

In the case of severe or complete blockage of a coronary artery, ischemia may exist even when the patient is at rest or engaged in minimal activity. In more moderate cases of coronary blockage, however, the reduced blood supply to the cardiac muscle may still provide sufficient oxygenation when the patient is at rest or engaged in moderate activity. However, when a patient engages in exercise at a significant level of workload, or is engaged in other activities which significantly increase the heart's demand for oxygen, the available blood flow through the partially blocked arteries may be insufficient to meet the increased demand for oxygenation. The result may be a transitory episode of cardiac ischemia, which subsides when the level of activity (and hence the demand for oxygen) decreases.

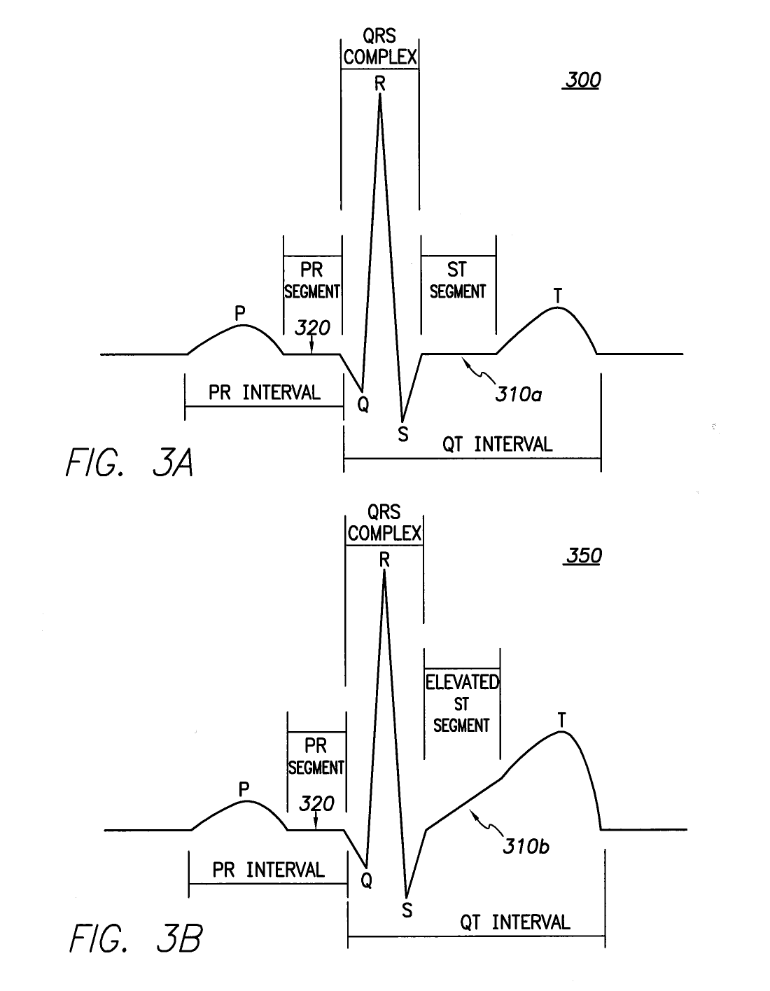

FIG. 3A and FIG. 3B together illustrate diagnosis of an episode of cardiac ischemia. FIG. 3A illustrates a representative view 300 of an ECG (also known as an EKG) or EGM, which may be obtained from a patient via an electrocardiograph or ICD 110, respectively, the ECG or EGM being a graphical representation of cardiac electrical activity. In an exemplary embodiment, the present method may obtain an EGM via an ICD 110; for brevity, the discussion here will refer exclusively to an EGM obtained via an ICD 110, it being understood that the present method may equally be implemented by obtaining an EKG via an electrocardiograph.

EGM 300 is indicative of the functioning of a heart which is receiving adequate oxygenation. The ST segment 310a represents the electrical activity during the interval between the ventricular depolarization (following ventricular contraction) and ventricular repolarization (in preparation for the next contraction). The ST segment 310 is substantially horizontal and substantially level or nearly level with the PR segment 320, indicating the cardiac tissue can maintain a membrane potential. This in turn indicates adequate tissue oxygenation.

FIG. 3B illustrates a representative view of an EGM 350 which may be indicative of cardiac ischemia. In particular, the elevation and sloping of the ST segment 310b is typically indicative of insufficient oxygen being delivered to the cardiac tissue. It should be noted that, depending on the exact location, extent, and degree of the ischemia, the nature of the ST segment elevation may vary. For example, in some ischemias, the ST segment 310b may still be substantially horizontal, but may be elevated above the level of the PR segment 320 to a degree that is medically significant.

As noted above, in an EGM the cardiac electrical vectors may vary depending on the pairs of leads used for signal measurement in the heart (for example, LVₜᵢₚ-can, RVₜᵢₚ-can, LV_{ring}-RV_{ring}, LVₜᵢₚ-RVₜᵢₚ, etc). As a result, in some instances ST segment depression (i.e., a lowering and/or downsloping of the ST segment), rather than ST segment elevation, may be indicative of ischemia. For convenience and brevity, in the discussion which follows reference is made primarily to ST segment elevation, and the accompanying figures illustrate ST segment elevation, as a determining indicator of cardiac ischemia.

It will be appreciated that the choice of ST segment elevation as an indicator of cardiac ischemia is by way of exemplary embodiments only. Those skilled in the relevant art(s) will appreciate that the present system and method, as described herein, may equally well be employed in contexts where a measurement of ST segment depression is indicative of cardiac ischemia. ST segment elevation and ST segment depression, possibly in association with an upslope or downslope of the ST segment, may also be referred to jointly as ST segment variation.

It should be further understood that while graphical views of an EGM 300, 350 are presented here for purposes of illustrating the present method for determining a degree of vascular blockage, it is possible to implement algorithms (via hardware, firmware, or software) which may automatically and analytically determine ST segment elevation or depression based on EGM data without any requirement for visual presentation or human interpretation of a plot or graph. Such algorithms may be implemented within an ICD 110, or may be implemented in an external computer or other health assessment instrumentation to which an ICD 110 has downloaded EGM data.

In one embodiment of the present invention, ST segment elevation or depression may be determined by first determining a reference height or amplitude. The reference amplitude may be, for example and without limitation, the amplitude of the QRS complex, where the amplitude may be measured from the value (e.g., the voltage) of the resting PR segment or ST segment (which are normally at the baseline of the EGM) to the voltage of the peak point (i.e., the R-point) of the QRS complex. Once the amplitude of the QRS complex is determined, a further determination may be made of ST segment elevation if the segment is elevated from its resting state voltage to some percentage value, for example, 10% or 20%, of the amplitude (in volts) of the QRS complex. Analogous criteria may apply to determining ST segment depression, but with a corresponding decrease in voltage.

In an alternative embodiment, ST segment elevation or depression may be determined if the ST segment is elevated or depressed by some specific voltage value. In a clinical setting, where an EKG may be shown on a strip of paper, an elevation of the ST segment by one or a few millimeters may be considered clinically significant. An algorithm for determining ST segment elevation or depression may be based on knowing how many measured volts (for example, 0.05 mVolts) may correspond to a two millimeter elevation on a physical strip of paper. The precise voltage may vary substantially depending on the voltages being detected by the ICD technology in use. Similar considerations, e.g., percentage change or absolute voltage values, may apply to using the slope of the ST segment as an indicator of ST segment elevation or ST segment depression.

More generally, the exact parameters used to determine ST segment elevation or ST segment depression, such as a degree of segment elevation or depression or a degree of segment slope, may be chosen in part based on criteria which may be found in the literature of the art, which may be established via clinical studies, and which may also be varied for purposes of setting different ischemia detection thresholds. The shape of the ST segment variation may also vary depending on lead vector variation.

### 5. Methods and Systems for Assessing Degree of Vascular Blockage by Measuring the Time between Onset of Workload and Onset of ST Segment Elevation

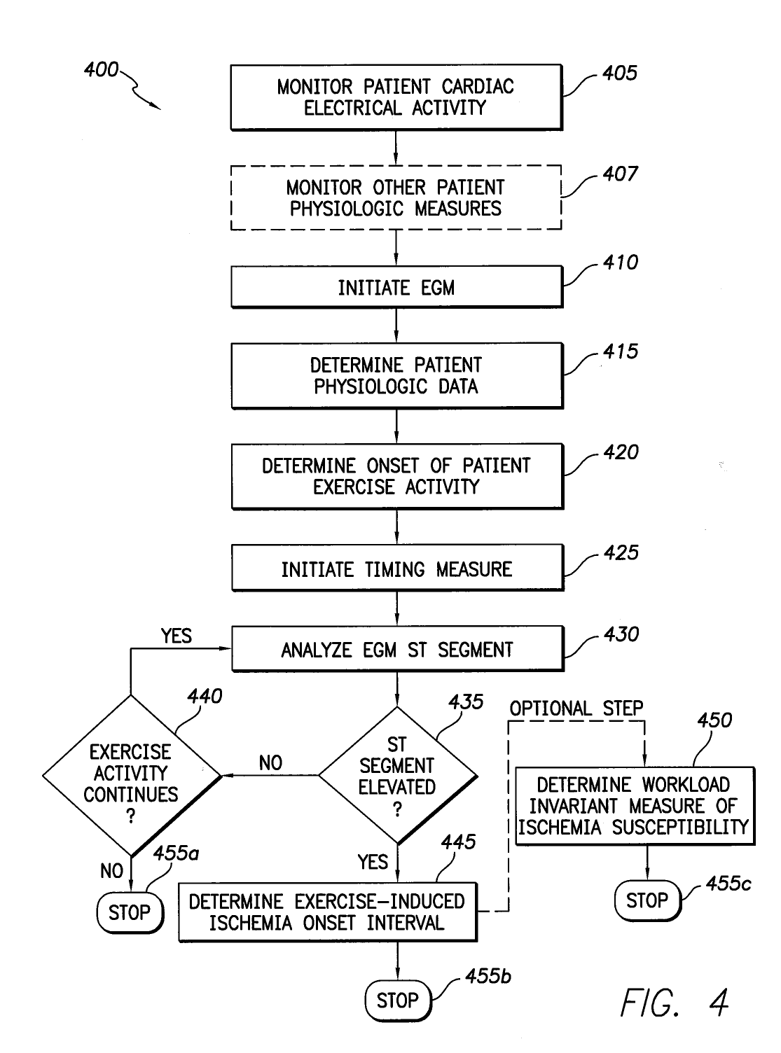

FIG. 4 illustrates an exemplary method 400 for making an assessment of a degree of vascular blockage or, equivalently, an assessment of ischemia susceptibility, by measuring the time between onset of an exercise workload and the onset of ST segment elevation, which may be an indication of myocardial ischemia.

Method 400 begins with step 405 wherein monitoring of cardiac electrical activity is initiated. As discussed above, cardiac monitoring may be done via an internal ICD or may be performed via an external electrocardiograph.

In optional step 407 (where the dotted lines indicate being optional), other physiologic measures may be initiated as well, such as measuring a patient blood pressure. A process of measuring a patient's motion, such as via an implanted accelerometer, may also be initiated.

Step 410 involves initiating an electrogram (EGM) or electrocardiogram (ECG or EGK), where an ECG is typically performed by an external electrocardiograph and the EGM would typically be performed by an ICD. The EGM or ECG is created based on the cardiac electrical activity monitored in step 405. The ECG or EGM may serve two purposes. One purpose is to assess the ECG or EGM for an elevated ST segment, which is indicative of an episode of ischemia. A second purpose may be to obtain measurements of pulse (i.e., heart rate) which may be used to determine an onset of patient exercise activity. An activity sensor, e.g., an accelerometer, may also be used to detect exercise.

In an exemplary embodiment, the present method may obtain an EGM via an ICD 110; for brevity, the remainder of the discussion will refer exclusively to an EGM obtained via an ICD 110, it being understood that the present method may equally be implemented by obtaining an ECG via an electrocardiograph.

In step 415, patient physiologic data is determined. As already indicated, some of this data, such as a patient pulse rate, may be obtained from the EGM. Other pertinent physiologic data, such as blood pressure, respiration rate, or other relevant physiologic factors may be obtained from other monitoring devices which may be associated with an ICD or other monitoring equipment.

In step 420, the onset of patient exercise activity is assessed. Typically, this is determined by assessing that some measure of the patient's metabolic activity or, equivalently, some measure of the patient's physiologic activity has passed a certain threshold. For example, pulse rate, respiration rate, or blood pressure may exceed a certain metabolic threshold, indicating that patient exercise activity has commenced. The metabolic threshold may also be referred to as an exercise threshold.

In general, there may exist a preferred threshold or workload level which is considered to be indicative of patient exercise. The precise level of this threshold level of patient exercise activity may vary depending on the individual patient, and may be a parameter which may be set as part of the process or method to determine the degree of vascular blockage. In general, a number of methods may be employed to determine when patient physiologic activity or patient movement has crossed an established threshold, thus indicating the onset of patient exercise activity.

In an exemplary embodiment of the present method, a composite physiologic signal such as a cardiac health index (CHI) may be used to determine when a patient is engaged in exercise. For a detailed discussion of determination of exercise via a composite physiological signal, which may combine heart rate, pulse rate, blood pressure, and possibly other physiologic signals, and possibly other indicators such as an accelerometer reading, see the commonly-owned, co-pending related U.S. patent application Ser. No. 11/611,105 to Steve Koh, filed Dec. 14, 2006, entitled “Exercise Compliance Monitoring and Benefit Assessment Via a Composite Physiologic Signal Determined by Implanted Physiologic Sensor”, the disclosure of which is incorporated by reference herein.

The present method requires that a determination be made of the time interval between the onset of patient activity and the assessment of an elevated ST segment. In step 425 timing is initiated. Typically, timing will be initiated at substantially the same time that a determination has been made that a patient is engaged in exercise activity. In one embodiment of the present method, timing may be initiated by storing in a database or other storage the time when exercise has begun. In an alternative embodiment, timing may be initiated by initiating a stopwatch or stopwatch-like measuring system, i.e., a timer, wherein the stopwatch is initiated when patient exercise activity commences.

Step 430 represents the initiation of an analysis loop in which the ST segment of the EGM is analyzed.

As discussed above, an elevated ST segment is considered to be indicative of an episode of cardiac ischemia. The exact parameters which are used to determine that the ST Segment is elevated above a normal or acceptable level, i.e., to determine an onset of cardiac ischemia, are based on criteria which are well known in the art and which furthermore may be fine-tuned to reflect individual aspects of an individual patient's cardiac condition. In general, the criteria may be indicative both of an absolute elevation of the ST Segment, and possibly of an increased slope of the ST Segment.

In step 435 an assessment is made as to whether the ST segment is elevated, which indicates the onset of a cardiac ischemic episode. If the answer is no, step 440 determines whether or not exercise activity continues. This assessment will be made using substantially the same metrics or analysis methods used in step 420 to determine the onset of patient exercise activity.

If patient exercise activity has ceased, then in step 455A the method stops. A recording may be made of the time when exercise activity ceased, or of the time interval between when exercise commenced and when exercise ceased. It may also be recorded that no episode of ischemia was detected during the interval, which may indicate either a healthy patient, or that the exercise interval was too short for a meaningful determination to be made of patient susceptibility to ischemia.

If in step 440 a determination is made that exercise continues, then the method returns to step 430 where an analysis of the EGM is again made. The loop continues in this fashion, assessing in step 435 whether there is an elevated ST segment—if not, then determining in step 440 whether or not exercise activity continues. If exercise activity continues, the loop through steps 430, 435, and 440 repeats as necessary.

If in step 435 a determination is made that there is an elevated ST segment, then in step 445 an exercise induced ischemia onset interval is determined. In one embodiment of the present method, this determination is made by comparing the time when exercise activity commenced and the time when the ischemia episode is recorded. In an alternative embodiment, the determination of the time interval may be made by stopping the timer which was initiated with the onset of patient exercise activity in step 425. Once the exercise-induced ischemia onset interval has been determined, this data may be stored in a short-term or long-term memory or database. In some embodiments, only a subset of the exercise-induced ischemia onset intervals may be stored, or other summary values, such as average exercise-induced ischemia onset intervals, may be stored.

Following step 445, step 455B stops the present method, meaning that an exercise-induced ischemia onset interval (EIIOI) has been determined and recorded. Alternatively, following step 445, optional step 450 may be performed. In step 450 a determination is made of a workload-invariant measure of ischemia susceptibility. The workload-invariant measure of ischemia susceptibility, which may also be known as a representative patient ischemia value, is discussed further below. Following step 450, the method stops at step 455C.

### 6. Methods of Analysis for Determining a Degree of Cardiovascular Blockage

It should be noted that in the discussion which follows as well as in the accompanying drawings, the Greek letter θ is used to represented a level of workload or level of patient exercise activity, which may be measured via any of several different specific metrics as discussed further below. Further, the Greek letter Δ is used to represent the length or duration of a time interval, and specifically a time interval between the onset of patient exercise activity and the detection of an ischemic episode in the patient. This same time interval Δ may otherwise be known as an exercise-induced ischemia onset interval, or EIIOI.

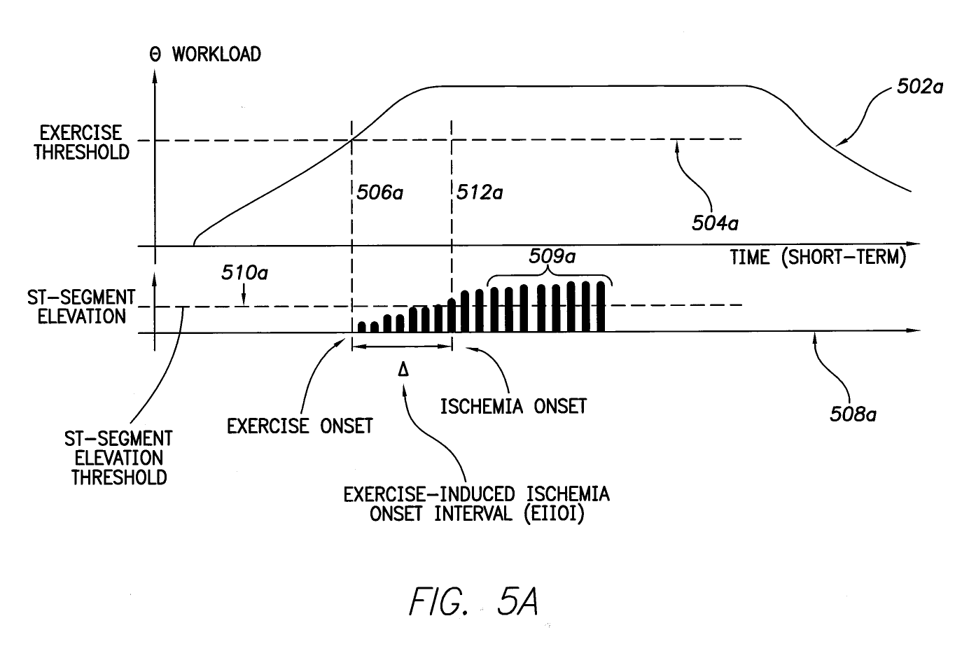

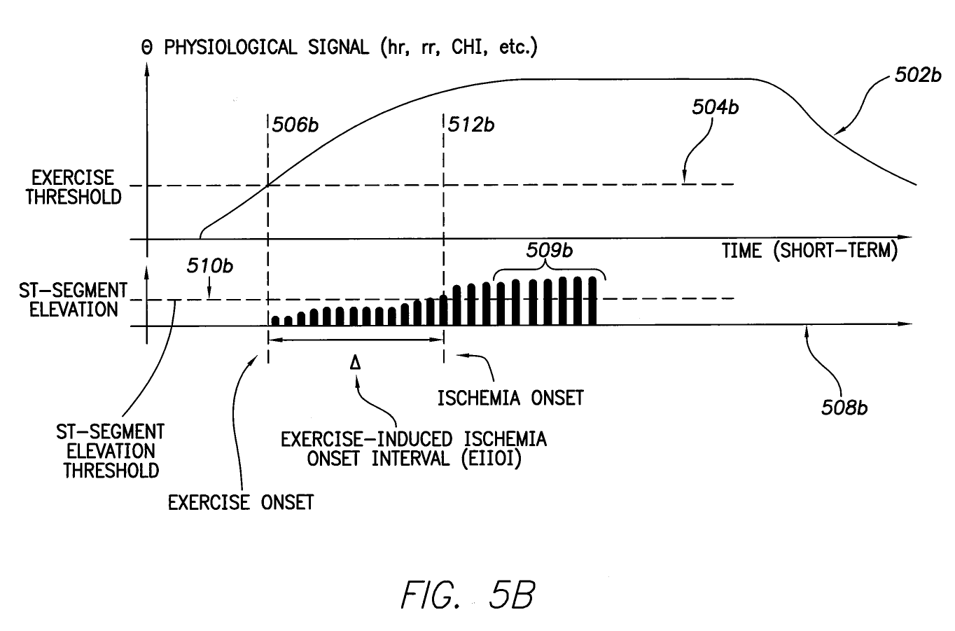

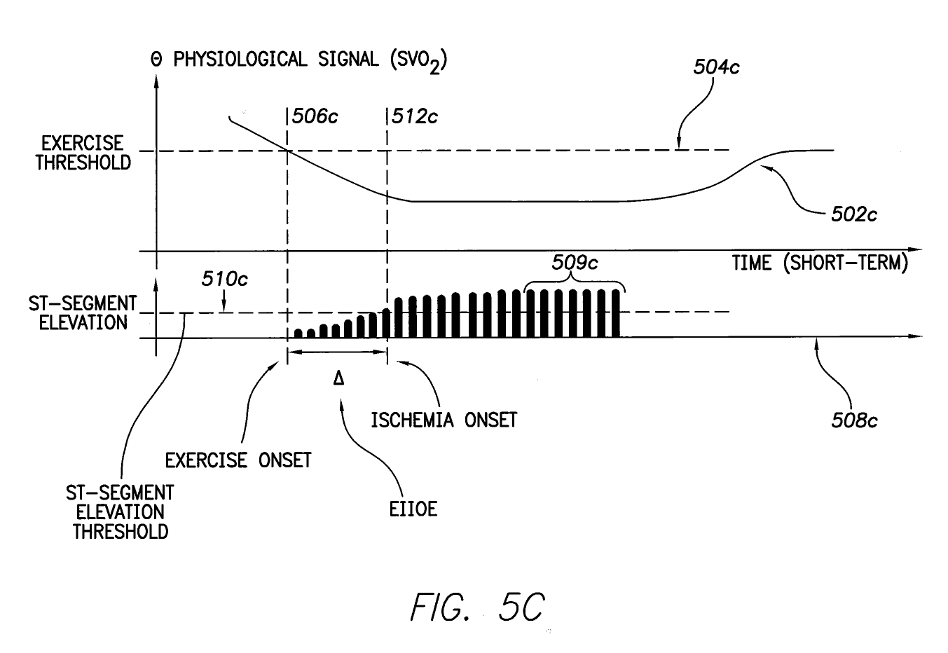

FIGS. 5A, 5B, and 5C each provide a graphical illustration of the method of the analysis for determining a degree of cardiovascular blockage.

FIG. 5A has two plots in parallel to each other, where a short-term time axis is in parallel in both plots, where short-term may be a matter of seconds, minutes, or possibly a longer time, and where the time scale of both plots is the same. The upper plot 502a indicates a level of workload, θ, which is experienced by a patient, where the workload θ may generally be construed as a level of exercise activity deliberately undertaken by the patient for the purpose of ascertaining a level of patient cardiac vascular blockage or risk of ischemia.

The plot line 502a indicates the level of workload or exercise activity which the patient is experiencing. This may be measured by a measure of physiologic activity, but may also be measured via an accelerometer or other device for detecting patient motion or activity. For example, and without limitation, the workload may be a level of difficulty on a treadmill, or a rate of cycling on a stationary cycle, or some other exercise workload or stress which is placed on the patient for the purpose of inducing an increase in cardiac activity. Horizontal exercise threshold line 504a indicates a level of workload at which it is considered that the patient has commenced exercise.

On the time axis is indicated a time of exercise onset 506a. Once exercise onset has begun, the EGM is analyzed to determine a level of ST segment elevation, shown by the thin vertical bars of ST segment elevation on lower plot 508a. When the level of ST segment elevation has crossed the ST segment elevation threshold 510a, it is determined that ischemia has been induced in the patient. This point in time is indicated by vertical line 512a.

A value delta (Δ) shown below the horizontal time axis indicates the time interval between line 506a and 512a, which is considered to be the exercise-induced ischemia onset interval (EIIOI). According to the present method, the shorter the EIIOI interval, the greater the likelihood of ischemia episodes for the patient, or the greater the degree of vascular blockage which may be responsible for ischemia episodes.

FIG. 5B is similar to FIG. 5A, but FIG. 5B illustrates that, as a matter of practical measurement, the level of exercise activity that is the workload level θ may be determined by assessing a physiologic signal such as heart rate, respiration rate, blood pressure, a composite health index (or CHI) which combines multiple physiologic signals, or similar value. The value of the physiologic signal or composite physiologic signal is reflected in plotline 502b. Not shown but possibly included may also be a value of an accelerometer rating or some other value which indicates a level of physical patient movement.

Once again, when the physiologic signal has crossed a certain exercise threshold level 504b, it is deemed that exercise has commenced, as noted by vertical line 506b. The ST segment elevation is then measured from that point forward on plot 508b, and as reflected in vertical bars 509b. When the ST segment elevation has crossed an ST segment elevation threshold 510b, it is concluded that exercise induced ischemia has occurred at time 512b.

The duration Δ between the onset of exercise 506b and the episode of ischemia 512b is the exercise-induced ischemia onset interval (EIIOI).

FIG. 5C illustrates an exemplary analysis method for determining a degree of cardiac vascular blockage in a patient. In this case the workload level θ, shown on plot 502c, is determined by a different physiologic signal, namely the internal blood oxygen concentration level, SVO₂. When this level, plotted on plotline 502c, decreases below an exercise threshold level 504c, this is taken as an indication that patient physiologic activity has increased to a point where exercise has commenced. The time is indicated by vertical line 506c. Once again a timing mechanism is initiated, which may involve the recording of the time of exercise onset, or the initiation of a timer, and the timing continues until the point of ischemia onset is reached. This point in time, indicated by vertical line 512c, is determined when the ST segment elevation values 509c shown on plot 508c cross the ST segment elevation threshold 510c.

The time interval Δ between exercise onset and ischemia onset is once again the exercise-induced ischemia onset interval, or the EIIOI. A longer EIIOI indicates that the patient was able to exercise for a longer period of time before the onset of ischemia. This may reflect a lower susceptibility to ischemia or, equivalently, a lower degree of coronary artery blockage. Similarly, a shorter EIIOI may reflect a higher patient susceptibility to ischemia or, equivalently, a higher degree of coronary blockage.

### 7. Workload-Invariant Measure of Ischemia Susceptibility

In order to determine a variation in a patient's cardiac health over an extended period of time, which may comprise weeks, months, or even years, it is desirable to be able to compare the degree of coronary blockage or, equivalently, the patient's susceptibility to ischemia, over time. Equally, it may be desirable to conduct empirical studies which may establish standardized metrics for a degree of coronary blockage or ischemia risk. Such studies may comprise determining the degree of ischemia vulnerability for patients in various age brackets, body weight/height classes, categories of prior medical history, etc.

Whether establishing comparisons between the same patient over a period of time, or comparisons between different patients, it may not always be practical to ensure that the same level of exercise difficulty or the same level of patient workload was employed when different measurements were taken. Hence, it is desirable to have a measure of vascular blockage or ischemia susceptibility which is substantially independent of the level of exercise workload required of the patient during testing. This may be known as a workload-invariant measure of ischemia susceptibility or, alternatively, as a representative patient ischemia value.

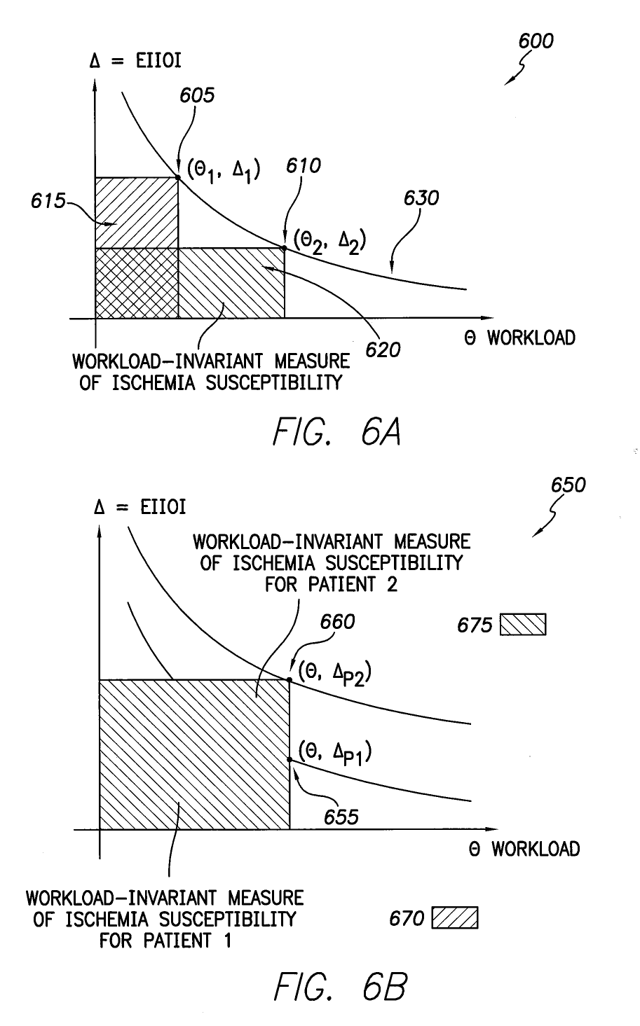

FIG. 6A illustrates a calculation of a workload-invariant measure of ischemia susceptibility (WIMIS). The measure entails taking a product of two values:

WIMIS=workload×EIIOI(=θ×Δ)

There are a number of options for the type of value used to represent the patient workload at the time the EIIOI was measured. In one embodiment, the workload may be measured based on an external measure of patient activity, such as a level of difficulty of a treadmill used by the patient during testing. However, because physical test equipment may vary, obtaining standardized measures in this way may prove difficult. A more robust measure of workload may be the threshold level of patient physiologic activity used to determine the onset of exercise. For example, the composite health index (CHI) discussed above may be employed. Other workload measures may be employed as well.

FIG. 6A illustrates how a WIMIS curve may be determined for a patient over a relatively short-term period of time, such as a few days or a few weeks, when it may be the case that the patient's health has neither improved or declined significantly. Over this relatively short-term period of time, the patient may be tested on several occasions to determine an EIIOI at different workload levels. The resulting sets of points (workload, EIIOI) or equivalently, (θ, Δ), may be plotted as shown in plot 600. In FIG. 6A, two such points 605, 610 are shown, which may reflect measurements made on different days. (Typically, it may not be desirable to perform two such tests at times very close to each other, such as within a few hours of each other, or within the same day, since a patient may need a time such as several hours, or possibly a day or more, to recover from the exertions of a single test.)

The product of the values θ1 and Δ1 of patient test point 605 is shown as the shaded area 615, which represents the WIMIS associated with point 605. Similarly, the product of the values θ2 and Δ2 of patient test point 610 is shown as the shaded area 620, which represents the WIMIS associated with point 610. Assuming the patient's degree of cardiac coronary blockage remains substantially unchanged over the short-term time between the two testing episodes, it may be seen from plot 600 that the two areas 615 and 620 may be expected to be substantially the same. It may further be expected that a plotline 630 of multiple such points may be expected to substantially conform to an equation of the form:

workload×EIIOI=WIMIS_{fixed}

where WIMIS_{fixed} is some fixed value.

It should be noted that in actual application, measured pairs of points (workload, EIIOI) may not have a product: workload×EIIOI which is exactly equal to WIMIS_{fixed}. Rather, it is expected that measured points will tend to cluster around a curve which can be represented by the equation:

workload×EIIOI=WIMIS_{fixed}

The curve or plotline associated either with this equation, or associated with a substantially reasonable match or substantially best-fit match to actually measured WIMIS points for a patient, may be known variously as a WIMIS curve or WIMIS plotline, an ischemia risk curve, an ischemia risk plotline, a vascular blockage assessment curve, a vascular blockage assessment plotline, or by similar terms.

It should be further noted that a patient may suffer from a sudden and potentially severe cardiac episode, which dramatically decreases cardiac performance. In these events, it may be the case that measurements of the WIMIS made shortly after the cardiac episode no longer substantially conform to the WIMIS_{fixed} value determined prior to the cardiac episode. Similarly, medications or other treatments which result in relatively sudden, and relatively dramatic improvements in overall cardiac health my result in relatively sudden increases in measured values for WIMIS_{fixed}. Therefore, the use of the calculation:

WIMIS=workload×EIIOI

as a workload-invariant measure of coronary vascular blockage should be employed with discretion, and with suitable considerations of patient clinical history in mind.

Finally, it should be noted that the WIMIS curve illustrated, and the associated equation, assumes that the actual level of physical exertion required by the patient will increase as the workload measure increases. This is typically the case for many of the possible workload measures discussed above, such as pulse rate, respiration rate, blood pressure, for many possible values of a composite health index (CHI), and for typical accelerometer readings.

However, some possible measures of patient workload, such as patient blood oxygen level, typically have lower values when the actual level of patient exercise increases. In such cases, another formulation or equation may be better suited to indicate a workload-invariant measure of ischemia susceptibility, or WIMIS. For example, if decreasing workload values, such as decreasing measures of blood oxygen concentration level, correspond to an actual increase in physical exertion by the patient, a suitable workload-invariant measure of ischemia susceptibility may be WIMIS=workload/EIIOI.

The remainder of the discussion below assumes that a metric for patient workload θ is being employed such that the equation:

WIMIS=workload×EIIOI(=θ×Δ)

is a suitable equation for determining a workload-invariant measure of ischemia susceptibility.

FIG. 6B shows a plot 650 which illustrates a comparison of the expected WIMIS curves for two different patients, Patient 1 and Patient 2. Patient 1, who may have the higher degree of coronary vascular blockage, may have a significantly lower or shorter EIIOI, as represented by the point (θ, Δ_{P1}) 655. Patient 2, who may have significantly lesser vascular blockage or possibly no vascular blockage has, at the same level of workload, a significantly longer interval period of time or EIIOI before the onset of ischemia, as shown at point (θ, Δ_{P2}) 660.

In FIG. 6B the WIMIS for patient 1, represented by the size of rectangular block 670 (shaded with diagonal hash marks from lower-left to upper-right), is significantly smaller than the WIMIS for patient 2, represented by the rectangular block 675 (shaded with diagonal hash marks from upper-left to lower-right). The lower WIMIS value for patient 1 indicates a significantly greater susceptibility to ischemia or equivalently, a significantly greater measure of vascular blockage, as compared to patient 2. (Note that the area of larger rectangular block 675 completely overlaps the area of smaller rectangular block 670, resulting in the appearance in FIG. 6B that rectangular block 670 is filled by crosshatching.)

Note that using the present method, a lower WIMIS value and a lower WIMIS curve represent a higher risk of ischemia or, equivalently, a higher degree of coronary vascular blockage. It will be apparent to one skilled in the art that alternative invariant or substantially invariant measures of vascular blockage could be developed based on the exercise-induced ischemia onset interval (EIIOI) of the present method; in some alternative embodiments, a higher risk of ischemia or, equivalently, a higher degree of vascular blockage, may be represented by a higher WIMIS value or analogous value, and/or a higher WIMIS curve or analogous curve.

### 8. Exemplary Methods of Assessing Patient Coronary Vascular Blockage or Degree of Ischemia Based on Standardized or Historical Data

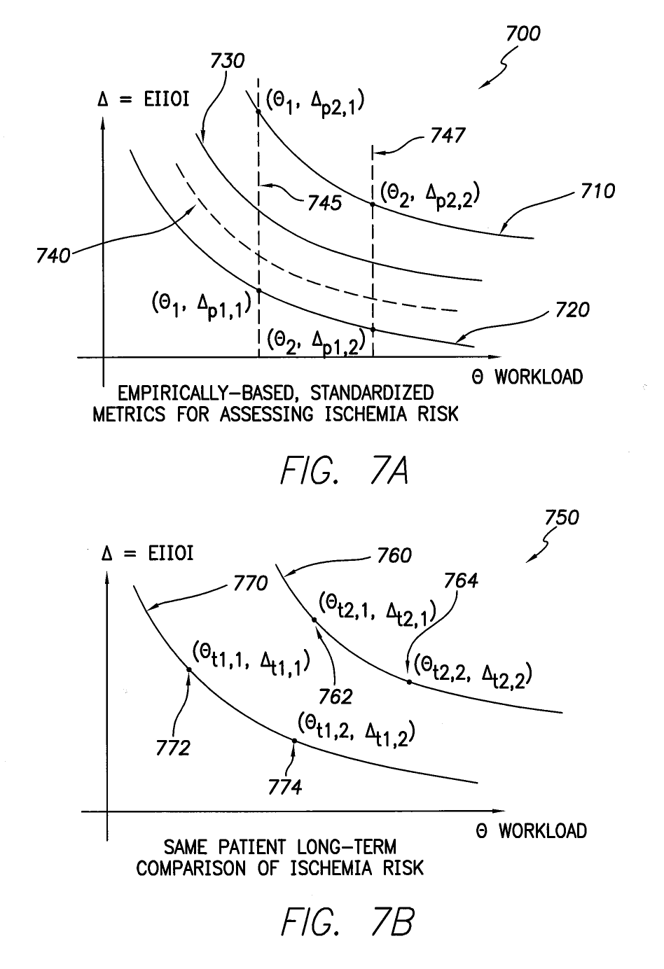

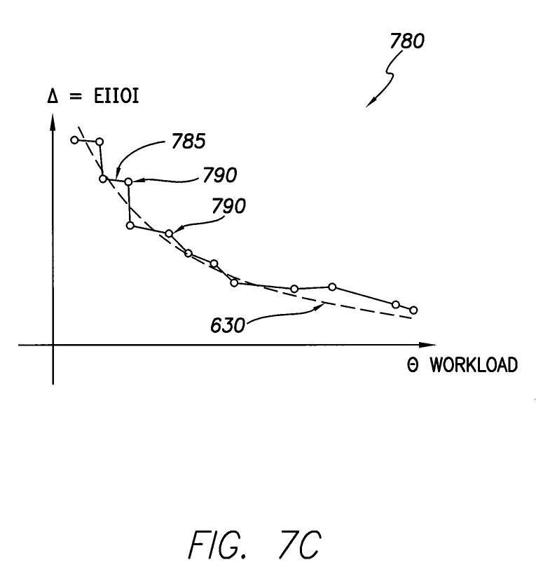

FIGS. 7A and 7B illustrate exemplary analysis processes by which assessments may be made of a degree of vascular blockage for a patient, based on prior historical data.

FIG. 7A illustrates an empirically-based standardized set of metrics for assessing vascular blockage or ischemia risk. These standardized metrics are based on historical cardiovascular data collected from a representative group of test subjects. A set of measurements may be conducted using a statistically significant assortment of test subjects to measure their degree of vascular blockage or their risk of ischemia, using the methods described above. For example, the workload-invariant measure of ischemia susceptibility (WIMIS) may be determined for each patient in a statistically representative group of patients by making measurements on separate occasions.

Using these measurements, a series of standardized WIMIS curves may be established. Three standardized WIMIS curves 710, 720, 730 are shown in FIG. 7A. An actual set of standardized metrics may have more curves. It should be further noted that the curves may be sorted onto separate plots and characterized by such other parameters as patient age, patient gender, patient weight and/or height, and other clinically pertinent factors which may serve to influence the placement or organization of the ischemia risk curves.

Once such standard WIMIS curves 710, 720, 730 have been created, possibly with associated clinical data from other testing modalities as described further below, they may be used for assessing a degree of coronary vascular blockage in other patients.

In plot 700, WIMIS curve 710 represents normal patients with no coronary artery blockage, WIMIS curve 720 represents patients who have a high degree of coronary artery blockage, and WIMIS curve 730 represents patients who have a moderate degree of coronary artery blockage. The WIMIS curve 740 for a current patient lies above curve 720 but below curve 730. This indicates that the current patient may have a higher degree of coronary artery blockage (and therefore higher risk of ischemia) than patients represented by WIMIS curve 730; and also that the current patient may have a lower degree of coronary artery blockage (and therefore lower risk of ischemia) than patients represented by WIMIS curve 720.

Plot 700 shows that in general, and for a given workload such as θ₁, the normal patients 710 who do not suffer from vascular blockage have a longer EIIOI than patients with vascular blockage, such as the highly ischemia-prone patients 720. Put another way, for the ischemia-prone patients, WIMIS curve 720 shows that the EIIOI is a shorter period of time at the same workload, as compared to normal patients 710 or patients with moderate risk of ischemia 730. This can be seen for example from inspection of the vertical EIIOI comparison lines 745 or 747.

For purposes of constructing such standardized metrics for assessing ischemia risk, the present method may be supplemented by other methods of determining vascular blockage including, for example and without limitation, MRIs, CAT scans, or other means of interior body scanning, blood testing to determine cholesterol levels, or other means which may be used to assess a risk of ischemia or a degree of vascular blockage.

FIG. 7B illustrates a means by which the degree of vascular blockage or ischemia risk for the same patient may be compared over a long-term period of time where a long-term period may be a period of at least several weeks, months, or years or longer periods of time. The time interval may also be somewhat shorter, if there exist clinical reasons to believe a significant change may have occurred in the patient's level of ischemia susceptibility over the shorter time frame.

Plot 750 has WIMIS curves 760 and 770. Curve 770 may be comprised of or defined by multiple WIMIS points, though in plot 750 only two exemplary WIMIS points (θ_{t1,1}, Δ_{t1,1}) 772 and (θ_{t1,2}, Δ_{t1,2}) 774 are shown. WIMIS points 772, 774 indicate measurements which were taken within a substantially short-term period of time, possibly within several days or possibly within several weeks of each other, or possibly within a few months of each other.

It is presumed that in most instances a patient will not experience a dramatic change in vascular blockage or a dramatic change in ischemia risk over such a short-term period of time, though such an assessment may ultimately rely upon a clinical determination made by a medical professional. Assuming the patient's coronary profile does not undergo substantial change within the short-term period of time, then measurements made within such a time frame would tend to fall on the same WIMIS curve. Therefore, for all the measurements just indicated (e.g., WIMIS points 772 and 774), the time period within which the measurements may be obtained is relatively short-term time period t1, which may be over days, weeks, or months.

During a relatively short-term second time period t2, which may extend over days, weeks, or months, the patient may be reassessed. Time period t2 may be a long-term period of time after t1, such as years later. Measurements during relatively short-term period of time t2 may yield multiple WIMIS points, such as exemplary points WIMIS points (θ_{t2,1}, Δ_{t2,1}) 762 and (θ_{t2,2}, Δ_{t2,2}) 764 on WIMIS curve 760.

In summary: WIMIS points (θ_{t1,1}, Δ_{t1,1}) 772 and (θ_{t1,2}, Δ_{t1,2}) 774 are two separate patient measurement points taken within the same relatively short-term time period t1; however they may be taken some short-term period of time apart, such as some days apart, and hence are points 1 and 2, which define in whole or in part WIMIS curve 770. Similarly, WIMIS points (θ_{t2,1}, Δ_{t2,1}) 762 and (θ_{t2,2}, Δ_{t2,2}) 764 also represent measurements taken at a significantly different time period t2 (e.g., many months or some years later than WIMIS points 772 and 774), but within a near term time frame to each other, and so define in whole or in part second WIMIS curve 760.

If the patient has been fortunate enough to have reduced vascular blockage at this later time t2, perhaps due to improved diet, exercise, medicine or surgery, the patient may now be at a lower ischemia risk as reflected by the higher level (i.e., indicating lower risk) of the ischemia risk curve 760 as compared with ischemia risk curve 770.

FIG. 7C provides another exemplary view of a WIMIS curve. In practice, the relationship between points on a single curve may not always be strictly invariant. An exemplary actual curve of WIMIS points, taken for example from a single patient over a relatively short period of time, may approximate an ideal curve, as shown on WIMIS plot 780. Here, exemplary actual WIMIS curve 785 may be approximately piecewise invariant (i.e., invariant or nearly invariant along portions of the curve), and points 790 of actual curve 785 may tend to cluster around exemplary ideal WIMIS curve 630.

It may typically be expected that WIMIS points from a single patient, taken during a relatively short time period, will tend to cluster around an ideal (i.e., workload-invariant) ischemia risk curve. Similarly, for standardized WIMIS metrics, WIMIS points representing a given population of patients with substantially similar clinical profiles and substantially similar levels of cardiac blockage may tend to cluster around an ideal (i.e., workload invariant) ischemia risk curve.

### 9. Further Exemplary Methods of Assessing Patient Coronary Vascular Blockage or Degree of Ischemia

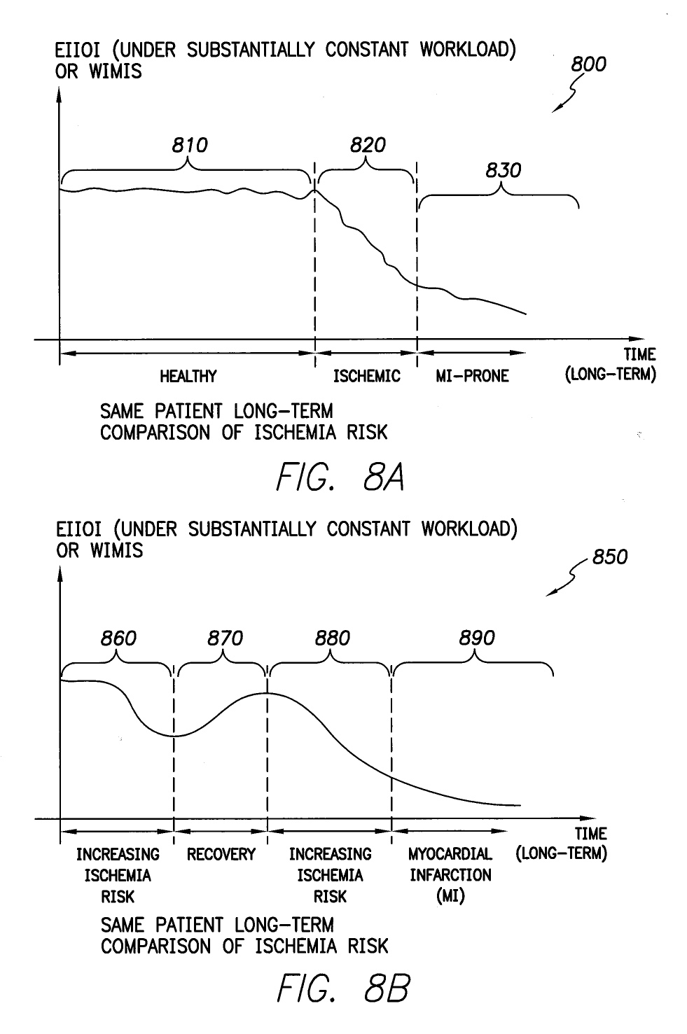

FIGS. 8A and 8B each illustrate an exemplary method of assessing a long term change in vascular blockage or ischemia risk for a patient. In either case, the assessment may be based on a workload invariant measure of ischemia susceptibility (i.e., on WIMIS values or similar or analogous measures), or based directly on the exercise-induced ischemia onset interval (EIIOI) itself.

If based on the latter (i.e., if the method relies upon the measured values for the EIIOI), then the method may assume that multiple values for a patient's EIIOI obtained over time have been determined based on a substantially constant or similar workload on each measurement occasion; the method may further assume that other factors, apart from a degree of coronary artery blockage, which may cause or induce transient variations in the EIIOI tend to substantially average out over time.

The timeframe for the analysis shown in both FIG. 8A and FIG. 8B is a long term timeframe which may be at least several weeks, but more likely months, years, or a longer term period of time.

FIG. 8A shows a plot 800 of measured exercise-induced ischemia onset intervals (EIIOIs) or WIMIS values as a function of time. During a first interval 810 when a patient is healthy, the EIIOIs or WIMIS values tend to have a high value. As the patient becomes progressively more ischemia prone or, equivalently, as there is an increased degree of vascular blockage, there is an interval 820 during which the EIIOI values or WIMIS values tend to decrease. Finally, if the patient becomes substantially myocardial infarction prone, meaning there is a highly significant and medically risky degree of vascular blockage, then there exists an interval 830 when the EIIOIl values or WIMIS values have a substantially lower value over an extended period of time.

FIG. 8B shows a plot 850 wherein a similar assessment is made over a long term period of time. In this case the difference is that there may be more variability over the long term period of time. During an initial interval 860 the decrease in the measured values for the EIIOI or WIMIS may indicate an increasing ischemia risk.

During a second interval 870, the trend towards increasing EIIOI values or WIMIS values tends to suggest a decrease in vascular blockage, which may be considered to be a recovery and an indication of improved cardiac health.

During a third interval 880 a decreasing trend in the values for the EIIOI or WIMIS once again indicate an increasing risk of ischemia or, equivalently, an increased degree of vascular blockage. Finally, during a fourth long term interval 890 the patient has very low values for the EIIOI or WIMIS, which may indicate a very high risk for myocardial infarction or may indicate that myocardial infarction has actually occurred and permanent damage may have been suffered by the patient.

While the discussion above has disclosed specific methods and systems to determine a degree of cardiac vascular blockage or risk of ischemia based on the time interval between the onset of exercise and an episode of ischemia, the scope of the present invention is not limited strictly to that disclosed. As just one example, it may be possible to augment the metrics discussed above by measuring and taking into account:

a degree of ischemia during an ischemic episode, as may be indicated by a degree of elevation of an ST segment or other indicators; and/or

a time interval for recovery from an ischemic episode, as again may be indicated by a degree of elevation of an ST segment or other indicators, and other physiologic and biometric factors as well.

More generally, it should be understood that, in addition to the specific means and methods specified above, a variety of means, methods, and systems may be employed within the scope of the present invention to:

determine a level of patient exercise activity;

determine a time of onset of patient exercise activity;

determine an episode of ischemia in a patient;

analyze an EGM or ECG for ST segment elevation as a means to determine the onset of an ischemic episode;

determine a time interval between the onset of exercise activity or other workload and the onset of an ischemic episode;

characterize a degree of cardiac vascular blockage or risk of ischemia in whole or in part in terms of the time between the onset of exercise activity on the onset of an ischemic episode;

characterize related metrics of cardiac vascular blockage or risk of ischemia, including but not limited to substantially workload-invariant measures of cardiac vascular blockage or risk of ischemia, based in whole or in part on the time between the onset of exercise activity and the onset of an ischemic episode, the level of patient exercise activity, and/or possibly on other biometric and physiologic factors;

make comparisons between the degree of cardiac vascular blockage or risk of ischemia between different patients based in whole or in part on the time between the onset of exercise activity and the onset of an ischemic episode, the level of patient exercise activity, and/or possibly on other biometric and physiologic factors; and

make long term assessments of the changes in a patient's degree of cardiac vascular blockage or risk of ischemia based in whole or in part on the time between the onset of exercise activity and the onset of an ischemic episode, the level of patient exercise activity, and/or possibly on other biometric and physiologic factors.

### 10. Conclusion

It is to be appreciated that the Detailed Description section, and not the Abstract section, is intended to be used to interpret the claims. The Abstract section may set forth one or more but not all exemplary embodiments of the present method and system as contemplated by the inventor(s), and thus, are not intended to limit the present method and system and the appended claims in any way.

What is claimed is:

1. A method for assessing a degree of cardiac vascular blockage in a patient comprising:
(a) determining an onset of patient exercise activity;
(b) determining an onset of myocardial ischemia; and
(c) determining a time interval between the onset of the patient exercise activity and the onset of myocardial ischemia,
wherein a shorter time interval indicates a greater degree of vascular blockage and a longer time interval indicates a lesser degree of vascular blockage.

2. The method of claim 1, wherein step (a) further comprises at least one of:
determining that a measure of patient metabolic activity has crossed a metabolic activity threshold;
determining that a measure of patient movement has crossed a patient movement threshold;
determining that a measure of external activity has crossed an external activity threshold; or
determining that a measure of a duration of exercise activity has crossed an activity duration threshold.

3. The method of claim 2, wherein determining that the measure of patient metabolic activity has crossed the metabolic activity threshold comprises measuring at least one of a patient heart rate, a patient blood pressure, a patient respiration rate, or a patient blood oxygen concentration level.

4. The method of claim 2, wherein determining that a measure of patient metabolic activity has crossed a metabolic activity threshold further comprises measuring the patient metabolic activity via an implanted physiologic measuring device.

5. The method of claim 2, wherein determining that a measure of patient acceleration has crossed an acceleration threshold further comprises measuring the patient acceleration via an implanted accelerometer.

6. The method of claim 1, wherein step (b) further comprises:
obtaining at least one of an electrocardiogram (ECG) or an electrogram (EGM) of the patient during the patient exercise activity; and
determining the onset of myocardial ischemia by determining an onset of at least one of:
an elevation of an ST segment in the ECG or EGM of the patient;
a depression of the ST segment in the ECG or EGM of the patient;
a change of slope of the ST segment in the ECG or EGM of the patient; or
a variation of shape of the ST segment in the ECG or EGM of the patient.

7. The method of claim 1, further comprising calculating a representative patient ischemia value, wherein the representative patient ischemia value is a product of the time interval and a level of patient workload;
wherein the level of patient workload is indicative of the level of patient exercise activity corresponding to the determination of the time interval; and
wherein if a first representative patient ischemia value for a patient and a second representative patient ischemia value for the patient have a substantially equal value, the substantial equality of the first and second values indicates a substantially similar level of ischemia for the patient.

8. The method of claim 1, further comprising determining a plurality of time intervals for the patient, wherein the plurality of time intervals are determined over a period of time, wherein the period of time is comprised of a plurality of days.

9. The method of claim 8, further comprising at least one of storing the plurality of time intervals, storing a subset of the plurality of time intervals, or calculating and storing a set of representative patient ischemia values, wherein each representative patient ischemia value is indicative of at least one of a patient time interval or a plurality of patient time intervals.

10. The method of claim 9, further comprising analyzing the plurality of stored values to determine at least one of a patient trend of cardiac vascular blockage over the period of time, a patient risk of ischemia, or a patient risk of myocardial infarction.

11. A method for assessing a degree of risk of cardiac ischemia in a patient comprising:
(a) determining an onset of patient exercise activity;
(b) obtaining a measure of patient cardiac electrical activity during the patient exercise activity;
(c) analyzing the measure of patient cardiac electrical activity to determine an onset of myocardial ischemia; and
(d) determining a time interval between the onset of the patient exercise activity and the onset of myocardial ischemia;
wherein a shorter time interval indicates a greater risk of cardiac ischemia and a longer time interval indicates a lesser risk of cardiac ischemia.

12. The method of claim 11, wherein step (a) further comprises determining that a measure of patient activity has crossed a respective activity threshold, wherein the measure of patient activity comprises at least one of a patient heart rate, a patient blood pressure, a patient respiration rate, a patient blood oxygen concentration level, or a patient acceleration; and
wherein the measure of patient activity is made via at least one of an implanted physiologic measuring device or an implanted accelerometer.

13. The method of claim 12, further comprising determining that the measure of patient activity has crossed an activity duration threshold.

14. The method of claim 11, further comprising:
obtaining at least one of an electrogram (EGM) of the patient or an electrocardiogram (ECG) of the patient; and
determining the onset of myocardial ischemia by determining an onset of at least one of:
an elevation of an ST segment in the ECG or EGM of the patient;
a depression of the ST segment in the ECG or EGM of the patient;
a change of slope of the ST segment in the ECG or EGM of the patient; or
a variation of shape of the ST segment in the ECG or EGM of the patient.

15. The method of claim 11, further comprising determining the onset of myocardial ischemia via an analysis of data obtained from an intra-ventricular pressure transducer.

16. The method of claim 11, further comprising calculating a representative patient ischemia value, wherein the representative patient ischemia value is a product of the time interval and a level of patient workload;
wherein the level of patient workload is indicative of the level of patient exercise activity corresponding to the determination of the time interval; and
wherein if a first representative patient ischemia value for a patient and a second representative patient ischemia value for the patient have a substantially equal value, the substantial equality of the first and second values indicates a substantially similar level of ischemia for the patient.

17. A system for assessing a degree of cardiac vascular blockage in a patient comprising:
means for determining an onset of patient exercise activity;
means for determining an onset of myocardial ischemia; and
means for determining a time interval between the onset of the patient exercise activity and the onset of myocardial ischemia; and
wherein a shorter time interval indicates a greater degree of vascular blockage and a longer time interval indicates a lesser degree of vascular blockage.

18. The system of claim 17, wherein the means for determining the onset of patient exercise activity comprises at least one of an implanted physiologic measuring device or an implanted accelerometer.

19. The system of claim 17, wherein the means for determining the onset of myocardial ischemia comprises an implanted physiologic measuring device, said implanted physiologic measuring device comprising a means of measuring a patient EGM;
wherein the onset of an episode of myocardial ischemia is determined by at least one of:
an elevation of an ST segment of the patient EGM;
a depression of the ST segment of the patient EGM;
a change of slope of the ST segment of the patient EGM; or
a variation of a shape of the ST segment of the patient EGM.

20. The system of claim 17, wherein the means for determining a time interval between the onset of the patient exercise activity and the onset of myocardial ischemia comprises at least one of a microprocessor of an implanted physiologic measuring device, an external microprocessor, an external computer, a timing circuit, or means of storing timing data.
